(19) United States
(12) Patent Application Publication
(10) Pub. No.: US 2014/0037708 A1
(43) Pub. Date: Feb. 6, 2014

(54) COMPOSITION FOR SPATIO AND/OR TEMPORAL RELEASE OF BIOACTIVE MATERIAL

(71) Applicant: The Regents of the University of Colorado, a body corporate, Denver, CO (US)

(72) Inventors: Wei Tan, Broomfield, CO (US); Walter Bonani, Livo Tn (IT)

(73) Assignee: The Regents of the University of Colorado, a body corporate, Denver, CO (US)

(21) Appl. No.: 13/954,789

(22) Filed: Jul. 30, 2013

Related U.S. Application Data
(60) Provisional application No. 61/678,017, filed on Jul. 31, 2012.

(51) Int. Cl. A61K 47/34; A61K 9/70

(52) U.S. Cl. CPC A61K 47/34; A61K 9/7007
USPC 424/426; 514/457; 514/8.2; 514/8.1; 514/8.9

(57) ABSTRACT

The present invention provides a biocompatible composition that comprises a bioactive material and a method for using the same. In particular, the composition of the invention is useful for spatio and/or temporal release of the bioactive material. Specifically, the composition of the invention comprises at least one polymeric mixture layer comprising a plurality of biodegradable polymers. The polymeric mixture layer comprises a first biodegradable polymer and a second biodegradable polymer. The relative amount of the first biodegradable polymer or the second biodegradable polymer increases within the polymeric mixture layer in one direction, and the relative amount of the other biodegradable polymer decreases in the polymeric mixture layer in the same direction. The rate of degradation of the first biodegradable polymer is different from the rate of degradation of the second biodegradable polymer. In this manner, the composition allows spatio and/or temporal release of the bioactive material.

### CROSS-REFERENCE TO RELATED APPLICATIONS

This application claims the priority benefit of U.S. Provisional Application No. 61/678,017, filed Jul. 31, 2012, which is incorporated herein by reference in its entirety.

### STATEMENT REGARDING FEDERALLY FUNDED RESEARCH

This invention was made with government support under grant number HL097246 awarded by the National Institutes of Health. The government has certain rights in the invention.

### FIELD OF THE INVENTION

The present invention relates to a biocompatible composition that comprises a bioactive material and a method for using the same. In particular, the composition of the invention is useful for spatio and/or temporal release of the bioactive material.

### BACKGROUND OF THE INVENTION

Electrospun polymer scaffolding has been used in a various tissue engineering. Such a technique provides biodegradable, high surface area matrix, with the opportunity of controlled bioactive material release. Exemplary bioactive materials that can be used in such polymers include drugs, proteins, oligonucleotides, chemical signaling agents, etc. Nanofiber surfaces provide high surface area aiding cell adhesion. In addition, high porosity of nanofibers aid in nutrient transfer and support maximal cell confluency. Scaffolding is a mechanical replacement of tissue before regeneration through production of extra-cellular matrix (ECM). Therefore, cell migration and proliferation, as well as polymer selection based on degradation rates are useful in scaffold design. Cell adhesion is influenced by inter alia surface area, which can be influenced through polymer selection and spinning parameters.

Chemical signaling can aid in cell proliferation and migration. Polymer fibers may be impregnated with biomolecules (i.e., bioactive materials), providing either surface release or through polymer degradation. Polymer choice provides not only control of biomolecule release rates, but control of diffusion through material properties and changing scaffold porosity also through degradation. Effective interplay between scaffolding materials and controlled release strategies is therefore useful. In general, scaffold functions can be extended to the delivery of signaling molecules either by using drug releasing scaffold or by incorporating drug delivery devices into the scaffold itself.

Bulk- and surface-eroding polymeric devices can serve as programmable biomolecule delivery systems to generate pulsatile release of one protein or sequential release of multiple biomolecules. Effective multipulse drug delivery system has been demonstrated using materials based on resorbable polyesters, polyanhydride-based laminates and crosslinked hydrogels. Some have developed PLGA scaffolds for spatially confined delivery and sequential release of VEGF and PDGF. Other have demonstrated the release of different bioactive molecules by sintering alternative layers of drug-loaded PLA microparticles plasticized with PEG and unloaded microparticles. In addition, PLGA microparticles have been used to obtain delayed release after an initial lag-time. Furthermore, several drug delivery materials have been recently developed for dual GF delivery. Still others have demonstrated dual release of GFs for neural regeneration by encapsulating PLGA microspheres loaded with NT-3 in a PLA-PEG-PLA hydrogel with free CNTF. Porous scaffolds have been shown to promote neo-vascularization as a result of the release of known pro-angiogenic GFs, such as VEGF and PDGF. Conventional compositions and techniques allow release of biomolecules without regards to any particular direction. In many instances, it is important to provide release of a biomolecule in a selective direction.

Accordingly, there is a need for a composition that can provide effective spatio and/or temporal release of bioactive molecules.

### SUMMARY OF THE INVENTION

Material layering through polymer co-spinning allows for programmed release of biomolecules over extended periods. The present inventors have discovered compositions and methods for spatially controlled biomolecule release using this method. Described herein is compositions and methods for achieving controlled spatio and/or temporal release of bioactive molecules. Polymer selection is one foundation of controlling diffusion during scaffold degradation. In one particular embodiment, poly-(ε-caprolactone) (PCL) and poly-(L-glycolic) acid (PLGA) were chosen for their degradation rates, with PCL exhibiting slow degradation and mechanical integrity over a period of months and PLGA exhibiting relatively accelerated degradation rate compared to PCL. Both polymers are considered hydrophobic, reducing diffusion in an aqueous environment, with PCL exhibiting higher rates of diffusion than PLGA. Co-spinning was established in creating solid-state chemical gradients through the control of solution feed-rates. Biomolecule concentration was controlled through embedment within PLGA during the co-spinning process. Faster degradation times compared to PCL allow for biomolecule release within the PCL scaffold, which can control chemical diffusion rates.

It should be appreciated that while PCL and PLGA are used herein for illustrative purposes, the scope of the invention includes use of other biocompatible polymers such as polyethylene glycol (PEG), various biocompatible polyesters and polycarboxylic acids, etc. Suitable biocompatible polymers can be readily determined by one skilled in the art having read the present disclosure.

Biomolecule diffusion within a static environment was monitored through custom diffusion cell. Nanofiber matrices were co-spun with fluorescent labeled albumin or rhodamine embedded within PLGA fibers. Patterning was controlled through the co-spinning process, with various levels of complexity in membrane design. Circular membrane samples were sealed in the radial direction with phosphate buffered saline (PBS). Diffusion was monitored through the fluorescence intensity of two solutions drawn from reservoirs on opposing sides of the sample membrane, perpendicular to the radial direction. Fluorescence measurement initially occurred every 24 hours for the first 15 days, and every 7 days subsequently, over 50 days.

Determination of chemical release rates using mathematical modeling can result in predictive design outcomes, minimizing design iteration. Diffusion coefficient for water (D) within separate PCL or PLGA nanofiber matrices can be determined experimentally. Diffusion within the scaffold will vary with composition, but is generally believed to vary linearly with PCL/PLGA ratio. Water uptake within the scaffold can be modeled by equation one:

$$\frac{\partial w}{\partial t} = \frac{d}{d x} ( {D}_{water} ( x ) \frac{d w}{d x} ) \qquad Eq . 1$$

This can then be extended with percolation theory to describe mass transport within the scaffold:

$$\frac{\partial {ρ}_{fix}}{\partial t} = - K {ρ}_{fix} \cdot w \qquad Eq . 2$$

where K is a constant dependent on the biomolecule released and the material of the scaffold, and w is the water uptake. Boundary conditions were set with closed boundaries in the radial direction, and open to size reservoirs limiting diffusion to one dimension spatially. Additional boundary conditions were modeled as D(x, w=0)=0, at t=0, for the diffusion function. Model outcomes were plotted against experimental values, and regression analysis completed.

Prolonged release of biomolecule signals is an important step in tissue engineering, aiding in cell infiltration over the duration of regeneration. Solid state chemical gradients aid in controlling diffusion rates, with material degradation responsible for changes in porosity and biomolecule release, and hydrophobicity reducing aqueous diffusion. Additionally, overall scaffold mechanical properties important in design may be maintained through the use of materials currently in use with tissue engineering. Patterning of differing nanofiber materials showed temporal control of biomolecule release for the purpose of tissue engineering scaffold design, displaying release times extending over days. Differing pattern gradients displayed prolonged release to specific, chosen directions. Regression analysis between experimental results and numerical models of simple, single molecule release patterns showed strong correlation.

Some aspects of the invention provide a biocompatible composition comprising:

a first surface;

a second surface; and

at least one polymeric mixture layer comprising a plurality of biodegradable polymers, wherein said polymeric mixture layer comprises a first biodegradable polymer and a second biodegradable polymer, and wherein the relative amount of said first biodegradable polymer or said second biodegradable polymer increases within said polymeric mixture layer in the direction from said first surface to said second surface, and the relative amount of said other biodegradable polymer decreases in said polymeric mixture layer in the direction from said first surface to said second surface, and wherein the rate of degradation of said first biodegradable polymer is different from the rate of degradation of said second biodegradable polymer.

It should be appreciated that the term polymeric mixture layer refers to two or more polymers that are separate but are mixed, often intimately, together. While each of the polymer can be a copolymer, which is formed as a single polymer from two or more monomeric units, the term “polymeric mixture” does not refer to a copolymer in and of itself.

In some embodiments, the composition of the invention can be described as comprising a combination of polymers A and B, and optionally a layer of polymer A, optionally a layer of polymer B. It should be appreciated that while the present invention is illustrated for two polymer system, the scope of the invention can include a plurality of different polymers. For example, compositions of the invention can include three, four, five, six, or more different polymers. It should be appreciated that the polymeric mixture can also include a plurality of biodegradable polymers. However, for the sake of brevity and clarity, the present invention is described herein in reference to a two polymer mixture system.

The polymeric mixture layer of the compositions of the invention is such that there is a gradient of polymer A (i.e., first polymer) increases in one direction and a gradient of polymer B (i.e., second polymer) increases in the other or opposite direction. Generally, the relative amount of polymer A increases from 0% to 100%, typically 0%-80%, often 0%-60%, and more often 0-50%. Of course, this means the relative amount of polymer B in the same direction will be 100% to 0%, 100%-20%, 100%-40%, and 100%-50%, respectively. However, it should be appreciated that the amount of polymer A need not start at 0%. For example, the relative amount of polymer A can increase from 10%-100%, 20%-100%, 30%-100%, etc. It should be appreciated that the scope of the invention includes polymer A gradient starting from 0% to 99%. Furthermore, the scope of the invention includes the upper limit of polymer A gradient starting from 100% down to 1%. Moreover, it should be appreciated that the polymer gradient need not be a linear increase. It can be a stepwise increase. For example, polymer A can increase at a rate of 0.1%, 0.2%, 0.3%, 0.4%, 0.5%, etc. up to typically 1%, often up to 5%, and often up to 10%. Such stepwise increase can be achieved by layering the polymeric mixtures. For example, a polymeric mixture of a particular thickness having a desired polymer A amount can be prepared and another polymeric mixture of a particular thickness having a different desired polymer A amount can be prepared on top of the first polymeric mixture. In this manner, a desired stepwise increment of polymer A can be achieved within a polymeric layer.

In some embodiments, the composition of the invention further comprises a plurality of said polymeric mixture layers. Such polymeric mixture layer can be dispersed within other polymer layers. For example, the first polymeric mixture layer can be separated from the second polymeric mixture layer by a layer of another one or more polymers (e.g., 100% polymer A or 100% polymer B layer). Moreover, one or more of the outer layers of biocompatible composition of the invention can include a layer of different polymer, such as 100% polymer A, 100% polymer B, or even a completely different polymer C. It should be appreciated that all outer layer polymer of the compositions of the invention are biocompatible polymers. Furthermore, all polymers are biodegradable such that the composition is degraded when placed in vivo to allow release of a bioactive molecule that is present within the polymeric mixture layer.

Yet in other embodiments, the biocompatible composition of the invention further comprises a barrier polymeric layer between two polymeric mixture layers. The barrier polymeric layer refers to a polymer or a polymeric mixture that does not include a bioactive molecule (i.e., biomolecule). In some instances, the barrier polymeric layer comprises the first biodegradable polymer, the second biodegradable polymer, a third biodegradable polymer or a mixture thereof.

In other embodiments, the first biodegradable polymer in the polymeric mixture layer comprises a first bioactive molecule. In some instances, the bioactive molecule is adsorbed in, attached to, or encapsulated within the first biodegradable polymer, or a combination thereof. In one particular instance, the first biodegradable polymer comprises covalently bonded bioactive molecule, non-covalently bonded bioactive molecule, or a combination thereof.

Still in other embodiments, the first bioactive molecule comprises a protein, a drug, an oligonucleotide, a chemical signaling agent, or other bioactive molecules known to one skilled in the art.

In some instances, the relative amount of the first biodegradable polymer decreases within said polymeric mixture layer in the direction from said first surface to said second surface.

Yet in other embodiments, the amount of said first bioactive molecule released towards said first surface during biodegradation is higher than the amount of said first bioactive molecule released towards said second surface. In some instances, the ratio of the amount said first bioactive molecule released towards said first surface compared to the amount of said first bioactive molecule released towards said second surface during biodegradation is at least 2:1, typically at least 3:1, often at least 4:1, and more often at least 5:1.

In one particular embodiment, the polymeric mixture layer comprises a nanofiber mixture of said first and second biodegradable polymers.

Still in other embodiments, said biocompatible composition is porous.

In other embodiments, the second biodegradable polymer comprises a second bioactive molecule. In such embodiments, when the first biodegradable polymer comprises a first bioactive molecule, the first and the second bioactive molecules are different from each other. In this manner, one can selective release the first bioactive molecule in one direction and release the second bioactive molecule in the other direction.

Other aspects of the invention provide a method for controlling the relative direction of and/or the relative rate of bioactive material released in a biodegradable polymer, said method comprising:

providing the bioactive material within a biocompatible polymer comprising:
a first surface;
a second surface; and
at least one polymeric mixture layer comprising a plurality of biodegradable polymers,
   wherein the polymeric mixture layer comprises

a first biodegradable polymer that comprises the bioactive material; and

a second biodegradable polymer,

and wherein the relative amount of said first biodegradable polymer or said second biodegradable polymer increases within said polymeric mixture layer in the direction from said first surface to said second surface, and the relative amount of said other biodegradable polymer decreases in said polymeric mixture layer in the direction from said first surface to said second surface, and wherein the rate of degradation of said first biodegradable polymer is different from the rate of degradation of said second biodegradable polymer.

### BRIEF DESCRIPTION OF THE DRAWINGS

FIG. 1 is a schematic illustration of the cross-section of a PCL-PLGA electrospun scaffold with internal compositional gradient. The fraction of PCL nanofibers (shown in blue) decreases along the thickness of the material from 1 on the left side (PCL side) to 0 on the right side (PLGA side), while the PLGA fraction (shown in red) increases along the thickness of the scaffold from 0 to 1.

FIG. 2 is design illustrations and confocal images of the gradient micropatterns over the thickness of PCL-PLGAac scaffolds. In the design illustrations (a, d) and confocal images (b, e), blue color shows PCL nanofibers loaded with fluorophore coumarin, red shows PLGAac nanofibers loaded with RhB, and green shows PLGAac nanofibers loaded with Rh123. The color intensity profiles (c, f), quantitatively determined from the confocal images, demonstrate the changes in the signal intensity of each fluorophore molecule across the sample thickness. Results demonstrate good agreement between the design patterns and the actual concentration profiles of the molecules or fibers.

FIG. 3 shows representative SEM micrographs of the PCL-PLGAac nanofibrous scaffold with an internal compositional gradient. Images were taken from the PLGA side. (a) Morphology of as-spun PLGAac nanofibers. (b-d) Changes in the nanofiber morphology after 1 week (b), 3 weeks (c), 5 weeks (d) of degradation in phosphate buffer solution at 37° C. The unchanged PCL nanofibers underneath the PLGAac layer were found after 5 weeks of degradation (d). Scale bar shows 10 μm.

FIG. 4 shows representative SEM micrographs of the PCL-PLGAes nanofibrous scaffold with an internal compositional gradient. Images were taken from the PLGA side. (a). Morphology of PLGAes nanofibers as spun. (b-f) Changes in the PLGAes nanofiber morphology after 1 day (b), 3 days (c), 1 week (d), 2 weeks (e), 4 weeks (f) and 6 weeks (g) of degradation in phosphate buffer solution at 37° C. The unchanged PCL nanofibers underneath the PLGAes layer were found in all these images (b-d). Scale bar shows 10 μm.

FIG. 5 shows GPC curves and molecule weight analyses of pure PCL, PLGAac, PLGAes materials. The weight-average molecular weight (Mw), number-average molecular weight (Mn) and polydispersity (pdi) are reported.

FIG. 6 shows GPC analyses of the as-spun PCL-PLGAac and PCL-PLGAes scaffolds and the scaffolds after hydrolytic degradation. (a-b) GPC curves of PCL-PLGAac and PCL_PLGAes; (c-d) Changes in number average molecular weight and remaining PLGA content determined by deconvolution of the GPC curves; (e-f) Overall remaining mass percentage of the scaffolds determined by the gravimetric method and measurement from GPC curves.

FIG. 7 shows the results of double-sided release test on PCL-PLGAac and PCL-PLGAes with Rh123 impregnated in the PLGA fibers. (a) Schematic illustration of the custom-made diffusion chambers with independently sealed reservoirs facing the two surfaces of the electrospun samples. (b) Release of Rh123 from the two surfaces of PCL-PLGAac scaffolds. (c) Release of Rh123 from the two surfaces of PCL-PLGAes scaffolds. The release was determined by measuring the fluorophore concentration in the chamber reservoirs. Herein, “Side B” indicates the fluorophore-loaded PLGA surface and “Side A” indicates the PCL surface.

FIG. 8 shows optical and confocal images of PLGA nanofibers prepared by one-phase, solution electrospinning Fluorecently-labeled, AlbF was dissolved directly in the polymeric solution and was homogeneously distributed along the nanofibers.

FIG. 9 shows the result of cumulative release of AlbT from the two surfaces of PCL-PLGAac composite scaffolds. The scaffolds were prepared following the compositional pattern presented in FIG. 1. AlbT was encapsulated in PLGAac and it was released mainly from the PLGAacrich side. “Side A” refers to the PCL-rich side, whereas “Side B” refers to the AlbT-loaded PLGAac side.

FIG. 10 shows the results of dual-drug release from one surface of the scaffold. (a) Illustration of the compositional pattern of nanofibers. (b) Cumulative release curve of AlbT. (c) Cumulative release curve of AlbF. (d) Comparison of AlbF and AlbT release during the first 10 days.

FIG. 11 shows the PCL-PLGA scaffold for the sequential release of proteins. (a) Illustration of the compositional pattern of nanofibers. AlbF-loaded PLGA nanofibers were confined in the middle of the scaffold, while AlbT-loaded PLGA nanofibers were close to side B; (b) Cumulative release profiles of AlbF and AlbT from the two surfaces of the scaffold; (c) Net release profiles of AlbF and AlbT to side B during the first 12 days

FIG. 12 shows the dual-drug release profile from the opposite surfaces of the PLGAac-PCL-PLGAac scaffold. (a) Illustration of the compositional pattern of nanofibers. (b) Cumulative release curve of AlbT. (c) Cumulative release curve of AlbF. Release of green-emitting AlbF was mainly confined to side A, whereas release of red-emitting AlbT is mainly confined to side B.

### DETAILED DESCRIPTION OF THE INVENTION

Controlled molecule release from scaffolds can dramatically increase the scaffold ability of directing tissue regeneration in vitro and in vivo. Useful in the regeneration of healthy functional tissues is regulation over release direction and kinetics of multiple molecules (small genes, peptides or larger proteins). To this end, one embodiment of the invention provides gradient micropatterns of electrospun nanofibers along the scaffold thickness through programming the deposition of heterogeneous nanofibers of poly(ε-caprolactone) (PCL) and poly(lactide-co-glycolide) acid (PLGA). Confocal images of the scaffolds containing fluorophore-impregnated nanofibers demonstrated close matching of actual and designed gradient fiber patterns; thermal analyses further showed their matching in the composition. Using acid-terminated PLGA (PLGAac) and ester-terminated PLGA (PLGAes) to impregnate molecules in the PCL-PLGA scaffolds, the present inventors have discovered their differences in nanofiber degeneration and molecular weight change during degradation. PLGAac nanofibers were generally more stable with gradual and steady increase in the fiber diameter during degradation, resulting in more spatially-confined molecule delivery from PCL-PLGA scaffolds. In some embodiments, patterns of PCL-PLGAac nanofibers were used to design versatile controlled delivery scaffolds.

To demonstrate that molecule-impregnated PLGAac in the gradient-patterned PCL-PLGAac scaffolds can program various modalities of molecule release, model molecules, including small fluorophores and larger proteins, respectively, were used for time-lapse release studies. Gradient-patterns were used as building blocks in the scaffolds to program simultaneous release of one or multiple proteins to one side or respectively to the opposite sides of scaffolds for up to 50 days. Results showed that the separation efficiency of molecule delivery from all the scaffolds with a thickness of 200 μm achieved >88% for proteins and >82% for small molecules. In addition to versatile spatially controlled delivery, micropatterns were also designed to program sequential release of proteins; temporal release kinetics was also altered by the nanofiber patterns. The hierarchically-structured compositions described herein can be used to develop various multifunctional scaffolds with defined 3D dynamic microenvironments for tissue regeneration.

Scaffolds made from nanofibrous materials using electrospinning techniques have been increasingly used for various tissue constructs, including those for replacement of intervertebral disk, meniscus, annulus fibrosus, blood vessels, and cartilage [1], [2], [3], [4], [5], [6]. The advantages of using nanofibrous scaffolds for tissue engineering include their load-bearing functionality, porous structure, and nanoto micro-sized fibers, similar in the length scale to native extracellular matrix (ECM), to guide cell adhesion and proliferation [7], [8], [9]. The versatile use of electrospinning with a variety of natural and synthetic degradable polymers offers a large repertoire for tissue engineering and drug delivery applications [10], [11], [12]. Additionally, electrospun biomaterials provide a wide range of mechanical and chemical properties via fiber composition, diameter, distribution and porosity, through control over a panel of engineering parameters in the process [12], [13]. Further, collection of nanofibers onto a rotating mandrel or other collectors with specialized surfaces, can result in structural and mechanical anisotropy for the applications of vascular or fibrous tissue engineering [14], [15].

In addition to structural support, mechanical property and biodegradable feature, an ideal scaffold for tissue regeneration should provide molecular cues to guide regeneration of tissue structure and function. To incorporate regenerative or therapeutic molecules into biomaterials that sustain the molecule release in vitro or in vivo, various methods such as adsorption and covalent binding, encapsulation, and addition during scaffold formation, have been developed. [16], [17], [18]. A step forward could be moving from static cues of one molecule toward delivery systems that reproduce more closely the dynamically evolving microenvironment occurring in natural ECM [19]. The delivery systems incorporating molecules, e.g., biodegradable particles, could offer distinctive advantages such as regulation of release rate while protecting their molecule cargo during all the stages of tissue regrowth [20], [21].

Scaffolds that support tissue repair, regrowth or regeneration from cells, particularly from undifferentiated stem or progenitor cells, ideally should partially recapitulate natural tissue morphogenesis, which is driven by the concomitant action of multiple factors working in concert. Growing evidence has shown that tissue morphogenesis is coordinated by the spatial arrangement and temporal duration of multiple molecules in the three-dimensional (3D) ECM [22]. Though the fundamental principles for regulating the assembly of cells into the functional tissues are far from being well understood, the main aspects which should be taken into account in delivery of growth factor or other molecules include: (i) concentration-duration relationship, (ii) stable concentration gradients, (iii) multiple factor delivery, and (iv) spatial patterning [23], [24], [25]. Recent advances in designing functional biomaterials attempt to harness some of these molecule regulatory mechanisms for tissue regeneration or other therapeutics [21], [25]. For controlled regulation, recent developments have demonstrated the benefits of using micro- and nano-structures [26], [27], [28], [29]. The majority of newly-developed molecule-impregnated scaffolds are capable of achieving sustained or temporally-controlled molecule release. Few techniques, however, are available to engineer 3D scaffolds that define spatial organization of molecules or control release in both space and time scale. Control of where a molecule acts can strongly contribute to provide spatially complex arrangements of cells in length scales ranging from nano-/micro-meters to centimeters. The spatial control over release is also critical to coordinate cell behaviors for tissue pattern formation [29], [21]. Selective release of molecules that target specific cell types or tissue formation without influencing the activity of other cell populations or tissue functions is important to establish both in vitro physiological tissue models and in vivo tissue regeneration [25]. Patterning or segregation of molecule delivery materials can be useful to spatially confine biomolecules [30]. To meet various physiological needs of different cell types during tissue regrowth, a 3D scaffold should present a multitude of regenerative biomolecules in a sustained manner at defined locations.

A challenging task is, however, to simultaneously build into a thin scaffolding material the desired control over molecular cues together with other functional requirements, including structural, mechanical, and degradable properties. Recently, strategies that use electrospun fibers to encapsulate and deliver molecules have been established [12], [13]. Due to inherently high surface area of electrospun materials, nanofiber constructs allow high molecule loading and efficient release in situ. But the developed techniques have been limited to tune molecule release kinetics and often the structure and property of nanofibers are significantly influenced to facilitate molecule encapsulation.

Described herein is a novel strategy of designing electrospun constructs, which utilize micropatterns of nanofiber to spatially or spatio-temporally control delivery of molecules. The present inventors have previously demonstrated the development of double-electrospinning system that used a rotating mandrel collector to produce constructs composed of interpenetrating networks of nanofibers with diverse nanofibers in a tailored proportion to achieve anisotropic mechanical properties and engineered surfaces [14]. Further developing the techniques to program micropatterns of molecule-impregnated nanofibers, the present inventors have discovered electrospun composite scaffolds that is capable of selectively releasing molecules in spatially- and temporally-controlled manner. In one particular embodiments, heterogeneous nanofibers composed of degradable polymers poly(ε-caprolactone) (PCL) and poly(lactide-co-glycolide) acid (PLGA), have been used to form scaffolds with compositional and biochemical gradients along the scaffold thickness. Using PLGA nanofibers to encapsulate small molecules or proteins, the present inventors have characterized compositional and morphological changes as well as degradation behaviors of these composite scaffolds for up to 50 days in vitro.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

### Materials

LACTEL® 50:50 poly(d,l lactide-co-glycolide) acid with a carboxylate end group and intrinsic viscosity of 0.67 dL/g, hereinafter referred to as PLGAac, was purchased from DURECT Corp. (Pelham, Ala.). PURASORB® 50:50 poly(d,l lactide-co-glycolide) acid with an ester end group and intrinsic viscosity of 0.32-0.48 dL/g, hereinafter referred to as PLGAes, was obtained from Purac Biomaterials (Lincolnshire, Ill.). PCL with a molecule weight of 80 kDa, chloroform (CF), N,N-dimethylformamide (DMF), methylene chloride (MC), methanol (MetOH), 1,1,3,3,3-hexafluoro-2-propanol (HFIP), sodium phosphate (NaH₂PO₄), sodium phosphate dibasic (Na₂HPO₄) and sodium azide were all purchased from Sigma Aldrich Inc. (St. Louis, Mo.). Polymers were used as received. Solvents were of analytical grade and used without any further purification. Isotonic phosphate buffer solution was prepared by mixing a 0.13 M solution of NaH₂PO₄ with 0.002% sodium azide and a 0.13 M solution of Na₂HPO₄ with 0.002% sodium azide, and then adjusting pH and osmolarity to 7.4 and 290-300 mosmol/L, respectively. Sodium azide was used to prevent bacterial contamination.

Fluorescent dyes, rhodamine 123 (Rh123) with green fluorescence, rhodamine-B (RhB) with red fluorescence, coumarin with blue fluorescence, as well as fluorescently-labeled proteins, fluorescein isothiocyanate conjugate bovine serum albumin (AlbF) with green fluorescence and tetra-methyl-rhodamine isothiocyanate conjugate bovine serum albumin (AlbT) with red fluorescence were all obtained from Sigma Aldrich. The molecule weight, chemical formula, excitation/emission wavelength and usage in this study of fluorescent dyes and fluorescently-labelled albumins are summarized in the following table:

Fluorescent dyes, fluorescent-labeled albumins properties and their uses

| | Molar Mass | | Excitation/ | |
|---|---|---|---|---|
| Dyes | [g/mol] | Chemical formula | Emission[nm] | Use |
| Albumin- | 66000/ | 7 to 12 moles of FITC per | 485/535 | Release |
| Fluorescein | 389.4 | mole of albumin | | |
| isothiocyanate | | | | |
| (Alb F) | | | | |
| Albumin-Tetra- | 66000/ | About 1 mole of TRITC | 560/590 | Release |
| methylrhodamine | 364.2 | per mole of albumin | | |
| isothiocyanate | | | | |
| (Alb T) | | | | |

### Preparation of Electrospinning Solutions

PCL was dissolved in 75/25 CF/DMF at a concentration of 10% (wt). For both PLGAac and PLGAes, the pre-spinning polymer solutions were prepared by dissolving the polymer in the mixture of HFIP and DI water with a ratio of 7:1 (vol) and a final concentration of 17% (wt). To visualize the compositional gradient across the thickness of electrospun scaffolds and to characterize the molecule release profile, fluorescent dyes or fluorescently-labeled albumins were added to pre-spinning solutions. Deionized (DI) water was used to dissolve fluorescent dyes. A solution of Rh123 or RhB with a concentration of 1 mg/ml was prepared with DI water, and then mixed with HFIP (7:1). Subsequently, the mixture was used to dissolve PLGAac and PLGAes. A PCL/coumarin pre-spinning solution was obtained by dissolving the polymer at a concentration of 8% (wt) in a ternary solvent mixture of MC:DMF:MetOH with a ratio of 5:3:2 (vol), where MetOH served as the carrier for coumarin. This was done by first dissolving 10 mg of coumarin in 300 μl of DI water to obtain the stock solution, then adding MetOH to the stock solution to obtain a 5 mg/ml solution of coumarin, and finally preparing the prespinning solution with the ternary solvent mixture. To prepare PLGAac nanofibers for in vitro release of fluorescent-labeled albumins, 5 mg of AlbF or AlbT was dissolved in 90 μl of DI water. Then, 630 μl of HFIP was added to the solution, forming a mixture to dissolve 220 mg of PLGAac, resulting in a 17% (wt) polymer solution. The overall content of albumin was 2.3% (wt) with respect to the mass of the dry polymer. During the entire process, the dye-containing solutions were always shielded from light to preserve the activity of fluorescent species.

### Double-Electrospinning Process

The electrospun composite with the compositional gradient of distinct materials across the composite thickness was prepared through programming the sequential and/or simultaneous electrospinning processes of PCL and PLGA on a rotating mandrel collector, using a modified double-electrospinning system previously described by the present inventors [14]. Briefly, a layer of PCL nanofibers, an intermixed layer of PCL and PLGAac (or PLGAes) nanofibers, and a layer of PLGA electrospun fibers were deposited in sequence on the grounded cylindrical aluminum collector. The design of the compositional patterns along the scaffold thickness was achieved by controlling 3D movements of a collector, in coordination with separately programmed spinning processes. The cylindrical collector, with 8 mm in diameter, rotated at a constant velocity by a brushless rotating electric motor (BLF230C-A, Oriental Motor US, Torrance, Calif.). Each solution was individually stored in a 5 ml syringe. The syringe was connected to a blunt-ended needle that served as a charged spinneret. Each spinneret placed at the opposite side of the rotating collector, were perpendicularly oriented with respect to the principal axis of the collector, and connected to a separate high voltage power supply (ES30P-10W, Gamma High Voltage Research Inc, Ormond Beach, Fla.). Both spinnerets moved in concert over an 18 cm path with a speed of 5 m/s along the collector mandrel which was under the control of a linear motorized stage (EZS3D025-C, Oriental Motor). The feed rate of each solution varied independently via separate syringe pumps (NE-300, New Era Pump Systems, Farmingdale, N.Y.) during the course of deposition. The electrospinning flow rates were determined beforehand for each solution in order to form defect-free nanofibers. These flow rates, 0.6 ml/hr for PLGA fibers and 1.1 ml/hr for PCL fibers, defined the maximum flow rates. The collector geometry, the concentration of polymer solutions, and apparent density of pure PCL and PLGA electrospun nets were used to establish a correlation between the feed rate and the deposition rate of each polymer per unit of surface area. Detailed information of the solution compositions and electrospinning parameters is listed below:

Characteristics of polymer/dye solutions and electrospinning parameters

| | | | | Dye | | | |
|---|---|---|---|---|---|---|---|
| | | | Polymer | content | | Work | Feed |
| | Loaded | Solvent | conc. | [%] | Voltage | Distance | rate |
| Polymer | dye | mixture | [% wt] | wt_{dye}/wt_{pol.} | [kV] | [cm] | [ml/h] |
| PLGAac | / | HFIP/water | 17 | / | 26 | 20 | 0.15-0.6 |
| PLGAes | | 7/1 vol. | | | | | |
| PLGAac | Rh123 | HFIP/water | 17 | ≈0.012 | 24 | 20 | 0.15-0.6 |
| PLGAes | RhB | 7/1 vol. | | | | | |
| PLGAac | AlbF | HFIP/water | 17 | 2.23 | 22 | 20 | 0.15-0.6 |
| | AlbT | 7/1 vol. | | | | | |
| PCL | / | CF/DMF | 10 | / | 26 | 24 | 0.3-1.1 |
| | | 3/1 vol. | | | | | |
| PCL | Coumarin | MC/DMF/MetOH | 8 | ≈0.05 | 20 | 20 | 0.3-1.1 |
| | | 5/3/2 vol. | | | | | |

The electrospinning process was performed at room temperature in open atmosphere with a relative humidity of 20-25%. The overall thickness of the electrospun nets was measured using a micrometer screw gauge. The design thickness of PCL-PLGA materials was 150 μm; samples with actual thickness outside of the 135-165 μm range were discarded. When fluorescent molecules were used, the entire apparatus was shielded from light to preserve the dyes. Electrospun samples were stored in a desiccator and kept in the dark.

### Characterization of Nanofiber Morphology

The morphology of the electrospun fibers was observed using a JSM-6480LV scanning electron microscope (SEM) (JEOL Ltd., Tokyo, Japan) at 5.0 kV. Samples were sputter-coated with gold prior to analysis.

### Characterization of Compositional Micropattern

Scaffolds loaded with fluorescent dyes were embedded in cryo-optimum-cutting temperature compound (Andwin Scientific, Schaumburg, Germany) or in paraffin for histology. A series of 15 μm-thick sections were obtained with a cryostat or rotary microtome for confocal imaging. The cross-sections of the nanofiber scaffolds loaded with fluorescent species were examined under a Zeiss LASER Scanning 510 confocal microscope equipped with AR, HE/Ne and diode laser sources (Carl Zeiss Inc, Jena, Germany). The images were used to assess the dye distribution in the nanofibers and along the thickness of the nets. Compositional profiles of scaffolds were determined by measuring the color intensity of dyes in the confocal images, using ImageJ software (http://rsbweb.nih.gov/ij/). Fluorescent profiles were obtained by averaging at least 6 profiles acquired from 3 different samples.

### Thermal Analysis

Thermal analyses were performed using a differential scanning calorimeter (DSC) (DSC 204 F1 Phoenix®, Aurora, Ill., USA) under flushing nitrogen (100 ml/min). Each sample was first heated from −110° C. to 120° C. with an incremental rate of 10° C./min and held at each temperature for 3 min. The sample was then slowly cooled to −110° C. with liquid nitrogen at a cooling rate of 5° C./min and reheated for a second scan to 120° C. at a rate of 10° C./min. For the PCL-PLGA composites, the glass transition of PLGAac and PLGAes were measured from the first heating scans. The transformation was clearly visible and was accompanied by an appreciable enthalpic relaxation effect due to aging of the polymer. Therefore, the onset point and inflection point were both reported. In the second heating scan, the glass transitions of PLGA were not visible, due to the melting peak of PCL. The peak temperature of the melting transition of PCL was recorded and melting peaks of PCL in pure PCL sample and PCL-PLGA samples were quantitatively determined by integration. The crystallinity degree of PCL (α_{PCL}) was determined by comparing the melting enthalpy of PCL (ΔH_{PCL}) for PCL nanofibers with the theoretical value of a sample with 100% crystalline PCL (ΔH_{100%}), which equals to 139.4 J/g. Thus, α_{PCL}=100*(ΔH_{PCL}/ΔH_{100%}). The melting enthalpy was determined by calculating the area of the endothermic melting peak. Under the assumption that melting enthalpy of PCL remained constant in PCL-PLGA samples, α_{PCL} was used to determine the fraction of PCL nanofibers.

### Hydrolytic Degradation Analysis

Hydrolytic degradation analyses were performed on electrospun scaffolds at 37° C. in phosphate buffer solution (pH=7.4) under constant mild agitation up to 6 weeks. For each electrospun net, specimens were immersed in 5 ml of phosphate buffer solution in separate test tubes. Triplicate samples for each experimental condition were withdrawn at a predetermined time, carefully washed in DI water and dried under vacuum in a dessicator for 48 hours. Then, the samples were weighed on an analytical balance. Using the gravimetric method, weight loss of each sample was then determined and averaged. Corresponding changes of nanofiber morphology were monitored using SEM micrographs. Molecular weights of PCL, PLGAac and PLGAes were determined by gel permeation chromatography (GPC) (GPCmax™, Viscotek, Houston, Tex., USA) with a differential refractive index (RI) detector (Viscotek 3580), and a set of Viscotek viscogel columns with THF as the eluent at 30° C. The analytical GPC was calibrated using monodisperse polystyrene standards. Molecular weights of the as spun scaffolds and those after each degradation time were measured. The GPC curves of the materials showed a bi-modal distribution. The overlapped peaks were thus deconvoluted into two separate asymmetric peaks with the least-square method and assigned to PCL and PLGAac (or PLGAes). The peak-fitting parameters were conditioned using the peak parameters of the parent materials. The molecular weights corresponding to individual deconvoluted peaks were also determined.

### Release Profile Characterizations

For molecule release characterizations, the circular samples with 24 mm in diameter were cut from PCL-PLGAac and PCL-PLGAes scaffolds loaded with fluorescent dye or fluorescently-labeled albumin. For each experimental condition, 3 samples were used for analyses. Scaffold samples were placed in a custom-made diffusion-test system in which a sample was sandwiched between two reservoir chambers. Each side of the electrospun scaffolds thus faced a different reservoir filled with 1 ml of buffer solution. The reservoirs used SecureSeal hybridization chambers (Grace Biolabs, Bend, Oreg.) which tightly sealed the surface. Each reservoir had small holes for sampling and changing solutions. The holes were covered with silicone sealing tap during experiments to prevent evaporation. Thus, the molecule movements between the reservoirs could only be through the pores of the electrospun materials. The release tests were performed at 37° C. Samples of buffer solutions in both reservoirs were collected every day during the first 16 days, and subsequently once a week for scaffolds loaded with fluorescent albumins. A shorter release experiment was performed in case of electrospun scaffolds loaded with Rh123. After samples were taken, the reservoir chambers were rapidly rinsed with DI water and refilled with fresh phosphate buffer at 37° C. Samples of 200 μl in triplicates were analyzed in 96-well microplates with a Victor 2 microplate reader (PerkinElmer, Santa Clara, Calif.) to quantify the released amount of Rh123, AlbF and AlbT. The concentration and net amount of released Rh123, AlbF and AlbT were determined using the respective calibration curves. For example, calibration curves for AlbF and AlbT with the respective coefficient of determination are shown in the table below. The geometry and porosity of the molecule-releasing scaffolds kept constant for all the experimental samples. For the designs with multiple molecules, as various fluorophores could be simultaneously released by the samples, the possibility of the overlap of the fluorophore emission spectra was examined; we could find no overlaps in the interested spectrum ranges. Additionally, we introduced a parameter to quantify the efficiency of separation (S) which shows the scaffold's capability of confining molecule release to one specific side of the scaffolds. For each type of molecule, the efficiency of separation between Side A and Side B, S_{molecule}_{_{—}}_{A-B}, was defined as the percentage of the molecule released on Side A over the total amount of the molecule released on Sides A and B during the entire experimental duration.

Table of calibration curves for AlbF and AlbT with the respective coefficient of determination.

| | | | |
|---|---|---|---|
| Alb F | x < 20000 | y = 639.9 x + 366.9 | R² = 0.998 |
| Alb F | 20001 < x < | y = −2.432 x² + 687.7 x + 327.4 | R² = 0.999 |
| | 60000 | | |
| Alb T | x < 20000 | y = 457.4 x + 389.3 | R² = 0.996 |

“x” is to the measured fluorescence intensity and “y” is the concentration of the molecule in solution expressed in μg/ml. The net mass released can be calculated taking into account the known capacity of the release chamber.

### Results

The present inventors have designed and produced electrospun scaffolds for controlled release of multiple active molecules. The present inventors have also produced electrospun scaffolds with variable composition and graded micro-patterns of nanofibers along the thickness of the scaffold. The present inventors have discovered that compositions of the invention are capable of confining the release of model proteins to one side of the materials. Disclosed herein also is a discovery by the present inventors that the spatial patterns of nanofibers has the capacity of programming release kinetics. By using the basic patterns developed before as building blocks, the present inventors have engineered and produced the scaffolds for various release patterns. This was done through modifying the distribution and proportion of PCL and PLGA nanofibers along the thickness of the scaffold. The newly designed scaffolds maintained the ability to localize the delivery of each loaded biomolecule and demonstrated the capability of controlling the release kinetics.

PCL-PLGA Scaffolds with Micropatterned Nanofibers Demonstrated Custom-Designed Compositional Gradients

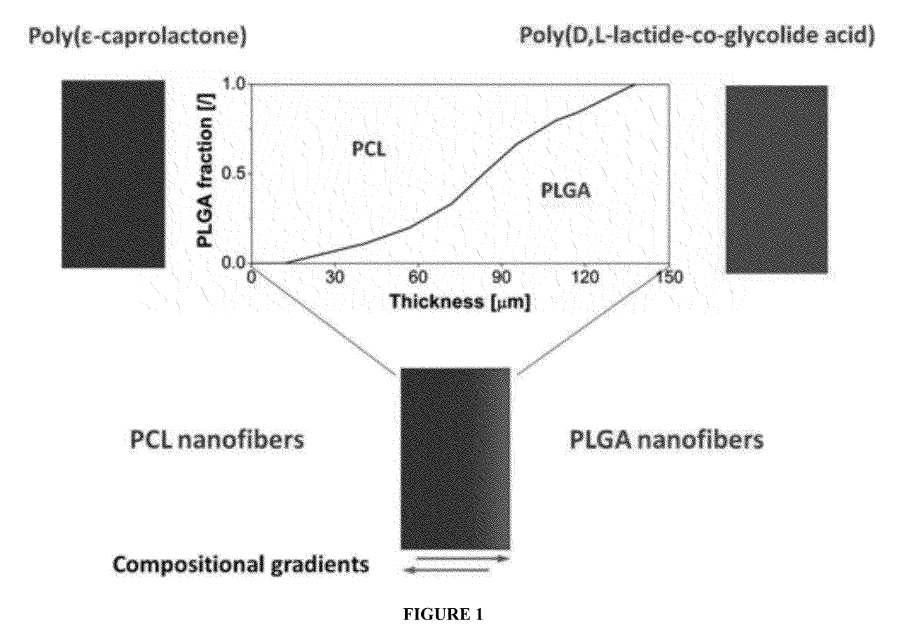

Using the double-electrospinning apparatus with the capability of programming the 3D position of different nanofibers, the present inventors have formed electrospun multi-component structures with custom-designed micropatterns. FIG. 1 illustrates a structure that consists of a 25 μm-thick PCL nanofiber layer, a 100 μm interlayer with mixed nanofibers of PCL and PLGA, and a 25 μm PLGA nanofiber layer. The design shows a pattern of pure PCL and PLGA on the surfaces of the opposite sides, and an intermediate region in which the PLGA content varies gradually between 0 and 100% along with a reversed change of the PCL content. This compositional gradient across the material thickness was achieved by gradually decreasing the feed rate of PCL to 0 while simultaneously increasing the feed rate of PLGA to the predetermined maximum value. The pattern was designed to produce scaffolds with equal amount of PCL and PLGA in weight.

To assess the actual composition of scaffolds, DSC was used. Results of DSC analysis curves were summarized in the table below:

Thermal properties of PCL and PCL-PLGA electrospun materials.

| | PCL | | | | | |
|---|---|---|---|---|---|---|
| | | ΔH_{PCL} | | | ΔH_{PCL} | |
| | Tm | melting | PCL | Tc | crystal. | PCL |
| | (II scan) | (II scan) | content | (cooling) | (cooling) | content |
| Sample | [° C.] | [J/g] | [% wt] | [° C.] | [J/g] | [% wt] |
| PCL | 60.0 | −58.5 | 100.0 | 27.1 | 55.9 | 100.0 |
| nanofibers | | | | | | |
| PCL-PLGAes | 61.3 | −31.9 | 54.5 | 27.9 | 29.7 | 53.1 |
| PCL-PLGAac | 59.1 | −32.2 | 55.0 | 26.7 | 30.5 | 54.6 |

| | PLGA | | | |
|---|---|---|---|---|
| | | | Tg | Tg |
| | PLGA content | PLGA content | (I scan, | (I scan, |
| | (from melting) | (from crystal.) | Onset) | Inflection) |
| Sample | [% wt] | [% wt] | [° C.] | [° C.] |
| PCL-PLGAes | 45.4 | 46.9 | 35.2 | 38.5 |
| PCL-PLGAac | 45.0 | 45.4 | 37.8 | 39.7 |

The results demonstrated that the actual material composition closely matched the design. The comparison between the endothermic melting enthalpies of PCL in the scaffolds and the melting enthalpies of pure electrospun PCL was used to determine the actual amount of PCL and the PCL/PLGA ratio in the scaffolds. PCL nanofibers showed crystallization enthalpy of 55.9 J/g during cooling and melting enthalpy of −58.5 J/g in the second scan. As the melting enthalpy of 100% crystalline PCL is 139.5 J/g, the resulting crystallinity of electrospun PCL nanofibers was 42%. Results from the crystallization and melting enthalpies of PCL-PLGAes and PCL-PLGAac scaffolds revealed that the actual PCL content was around 55% in both cases. Therefore, the actual PLGA contents were around 45%. Also, the glass transition temperatures of PLGAac and PLGAes, determined from the inflection point in the first DSC scan curves of PCL-PLGA scaffolds, were 39.7 and 38.5° C., respectively. The present inventors have also observed that the glass-rubber transition for PLGAes at the onset point started at 35.2° C., below the working temperature of 37° C., while the transition zone for PLGAac started at 37.8° C., just above the working temperature.

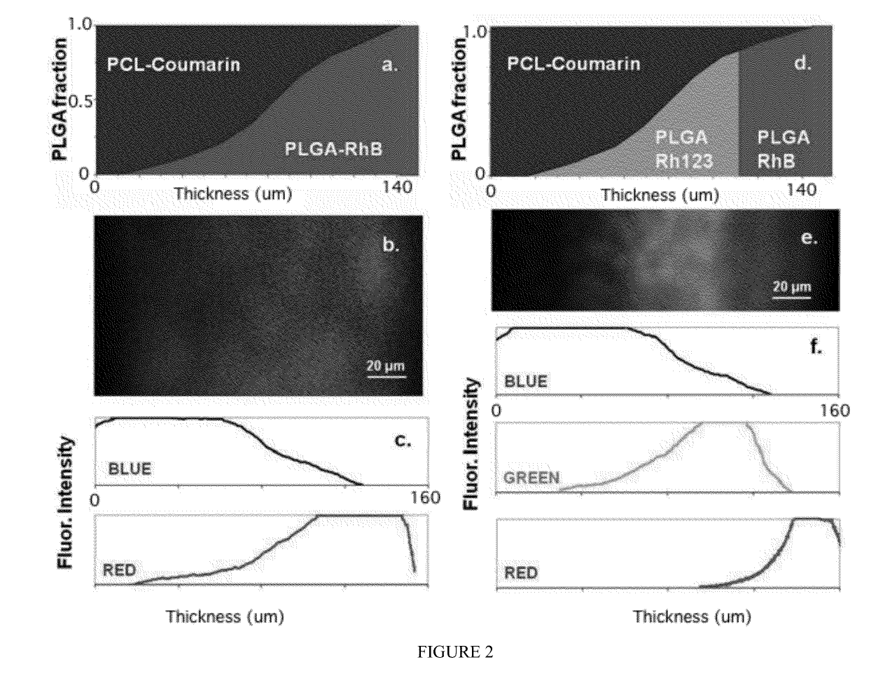

In addition to the closely-matching composition, confocal images of the cross-sections of molecule-impregnated nanofiber materials further demonstrated that the compositional gradients of impregnated molecules matched the designed nanofiber micropatterns (FIG. 2). Herein, PCL nanofibers were loaded with a blue fluorophore coumarin, while PLGAac nanofibers were loaded with red fluorophore RhB and/or green fluorophore Rh123. FIG. 2 demonstrated the molecule patterns in a two-component scaffold and a three-component scaffold. FIG. 2a showed the confocal image of a PCL-PLGAac material, in which PCL and PLGAac were loaded with blue and red fluorophores, respectively. The gradual increase in the red color intensity from the left to the right side of the image demonstrates the increase of PLGAac content across the material thickness, whereas the attenuation of blue intensity shows the reduction of the PCL fraction in the same region. Also, no red signals were found close to the surface on the PCL side, and no blue signals close to the surface on the PLGAac side. This reflects the pure polymer design in these regions. However, due to the saturation of fluorescent signals, the fluorescent profiles in the high intensity range could not be assessed with absolute certainty. FIG. 2b illustrated the design of a three-component scaffold, where PLGAac nanofibers were used to impregnate two molecules in the respective regions: green Rh123 in the inner region and red RhB in the outer region of the scaffold. Similar to the pattern observed in the two-component scaffold, the fluorescent intensity profiles in the confocal image reproduced the designed micropattern across the scaffold thickness with accuracy except in the high intensity range where the fluorescent signals saturated beyond detection.

### Hydrolytic Analyses of PCL-PLGA Scaffolds Demonstrated the Different Degradation Behaviors and Morphological Changes of PLGAac and PLGAes

To compare the hydrolytic degradation behaviors of PCL-PLGAac and PCLPLGAes scaffolds, samples were analyzed with the gravimetric method for weight loss, GPC for changes in the polymer molar weight distribution, and SEM for the nanofiber morphology. These results demonstrated the significant differences between PLGAac and PLGAes during the 6-week hydrolytic degradation process.

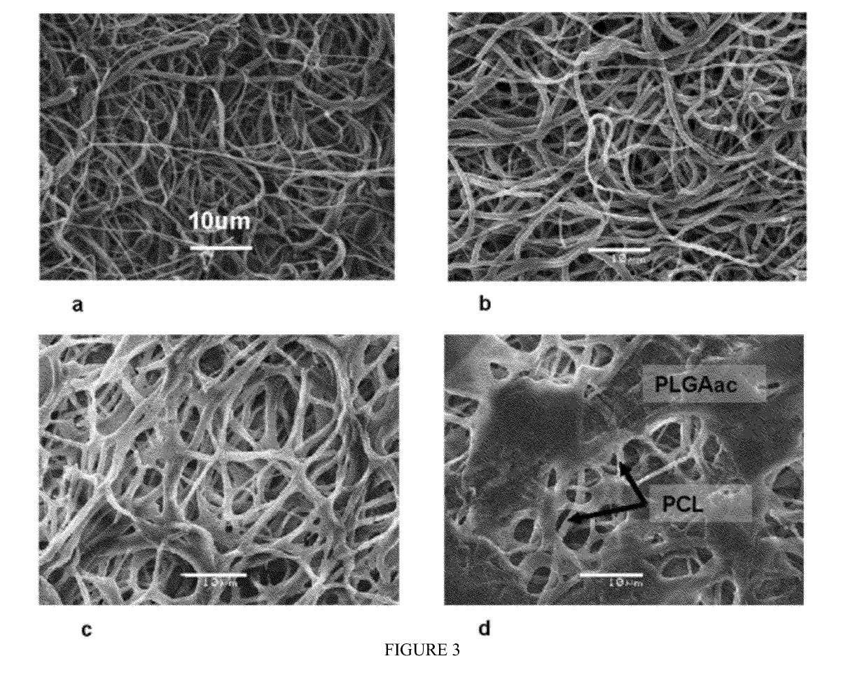

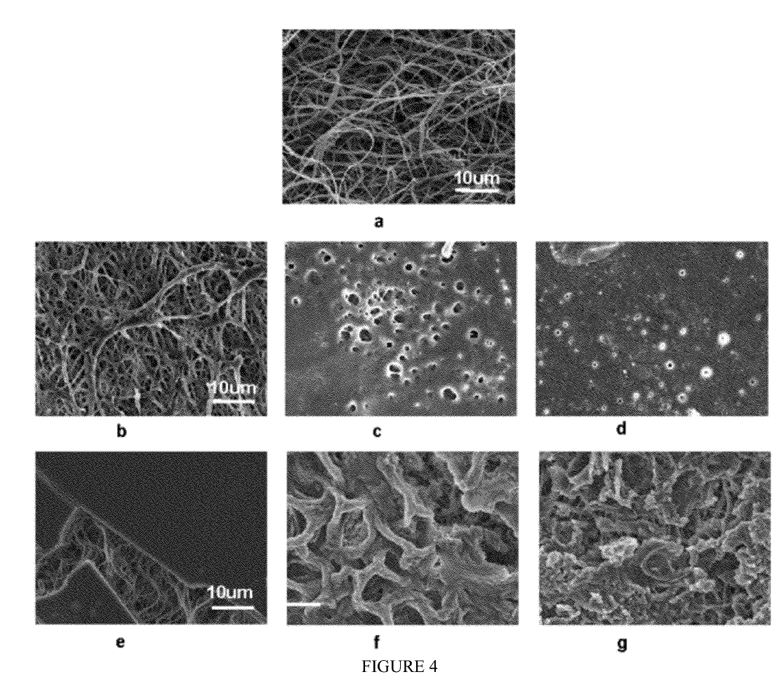

The SEM micrographs shown in FIGS. 3 and 4 demonstrated the evolution of PCL-PLGAac and PCL-PLGAes scaffolds during degradation. FIGS. 3a and 3b showed that the morphology and network of PLGAac nanofibers remained virtually unchanged after 1 week of degradation in buffer. During this period, the average fiber diameter increased about 25%, from 0.62±0.27 μm to 0.78±0.28 μm. After 3 weeks, PLGAac nanofibers exhibited more significant increase in diameter with an average diameter of few microns, and the fiber network was characterized by soldering-like attachments at the junctions (FIG. 3c). The SEM images also showed that by week 3, some fibers on the sample surface had lost their smooth cylindrical morphology. Also, the PCL nanofibers started to emerge in the SEM images; they were underneath the PLGA fibers, exhibiting unaffected cylindrical and smooth morphology. Overall, the deformed and swollen PLGAac nanofibers seemed to be collapsed onto, and wrapped around, the more stable PCL fibers after 3 weeks of degradation. After 5 weeks, the fibrous structure of PLGAac almost disappeared, and a film of PLGAac covered the web of PCL nanofibers which were unscathed after degradation (FIG. 3d). From a macroscopic view, it was found that the PCL-PLGAac scaffolds remained flat and dimensionally stable in terms of the overall geometry and size, during the entire experimental period.

Compared to PLGAac, the morphology of PLGAes nanofibers altered much faster (FIG. 4). After only 1-day exposure to buffer, the electrospun PLGAes nanofiber network started to collapse with extensive soldering-like attachments at the junctions (FIG. 4b). Within one day, the average fiber diameter increased nearly 80%, from 0.50±0.17 μm to 0.88±0.10 μm. After 3 days, no fiber-like morphology remained in the PLGAes layer. The layer turned into a membrane with only an array of small circular open pores (FIG. 4c) whose diameter continued to decrease in the first week (FIG. 4d). At the end of the second week, the PLGAes layer was entirely re-structured and became a solid substrate without pores lying on the bed of PCL nanofibers. FIG. 4e demonstrated the structure of PCL fibers lying under a fractured portion of the PLGAes layer. From then on, the surface of PLGAes layer appeared to be continuously eroded by the buffer, and after 6 weeks, a significant amount of material still remained on the surface (FIG. 4f-g). Similar to PCL-PLGAac, in the PCL-PLGAes composites, the morphology and network structure of the nanofibers on the PCL side remained unchanged during the degradation process. From a macroscopic view, it was found that the PCL-PLGAes scaffolds shrunk and deformed bending toward the PLGAes side, which might be due to the loss of PLGAes pores reducing the volume of the PLGAes material.

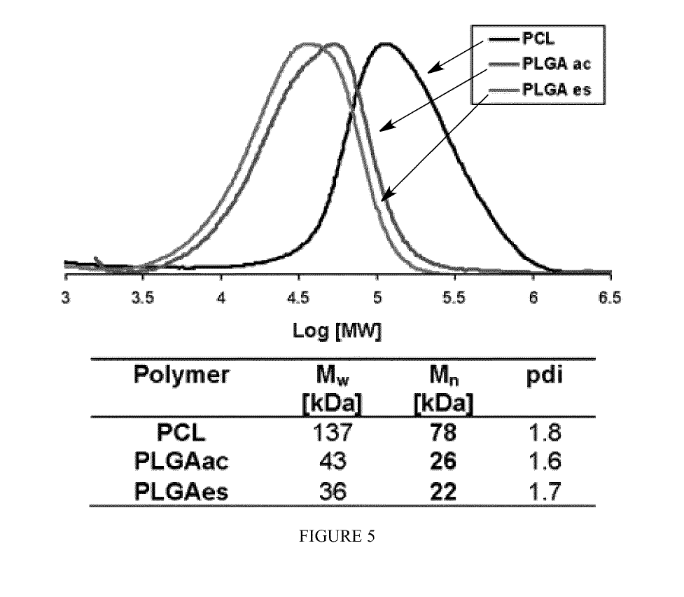

To gain further understanding of the hydrolytic degradation processes of the composite scaffolds, changes in the overall weight and molecular weight of different PCL-PLGA scaffolds were characterized. Using GPC curves of the component polymers (FIG. 5), the weight-average molecular weight (Mw), number-average molecular weight (Mn) and polydispersity (pdi) of pure PCL, PLGAac and PLGAes materials were determined. These data served as the basis for analyses of the PCL-PLGA composite degradation processes.

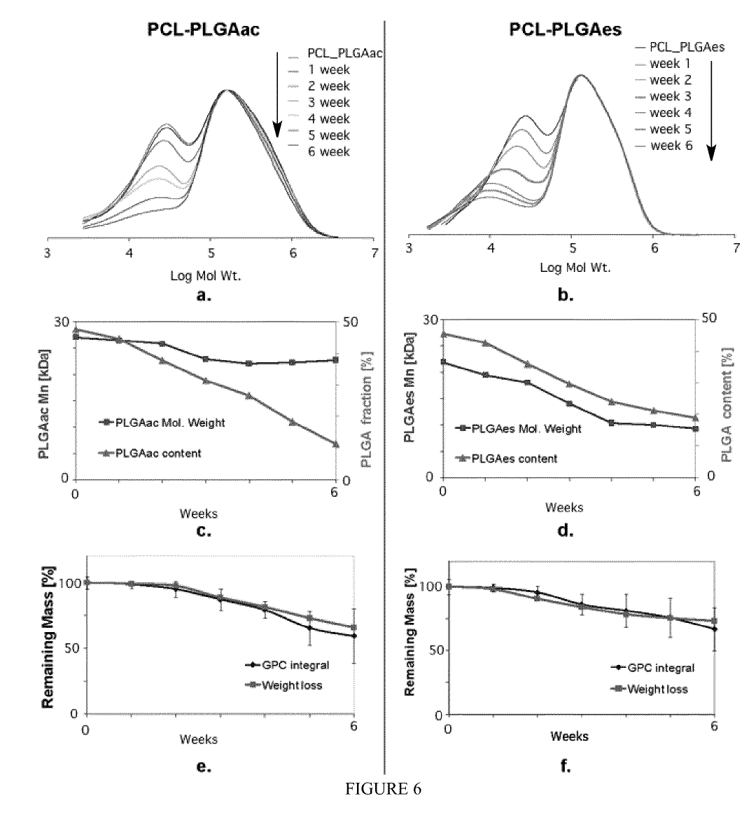

FIG. 6 demonstrated the differences between PCL-PLGAac and PCL-PLGAes scaffolds in the GPC curve, molecular weight change and weight loss during hydrolytic degradation. Both GPC curves exhibited a bimodal distribution. The peak at higher molecular weights was assigned to PCL, whereas the peaks at lower molecular weights were assigned to PLGA. GPC results from both PCL-PLGAac and PCL-PLGAes materials (FIG. 6a-b) showed that the PLGA peak gradually decreased and shifted towards lower molecular weights, while the PCL peak remained unaffected during the hydrolytic degradation. Using deconvolution analysis of GPC curves, the contributions of PCL and PLGA, the PLGA content in the scaffold and the number average molecular weight of the polymer were determined. As shown in FIG. 6c, the molecular weight of PLGAac slightly decreased between week 2 and week 3 before getting steady at 21 kDa. The PLGAac content in the scaffold steadily dropped to approximately 22% of the original amount after 6 weeks. Compared to PCL-PLGAac, the molecular weight of PLGA in PCL-PLGAes scaffolds dropped more progressively from 22 to 9 kDa, suggesting molecule breakdown during the experimental period (FIG. 6d). The PLGAes content decreased accordingly. However, when compared to PCL-PLGAac, there was a higher amount of PLGA content present in PCL-PLGAes scaffolds after 6 weeks, approximately 40% of the original amount of polymers. The weight loss curves for the PCL-PLGAac and PCL-PLGAes scaffolds were presented in FIG. 6e and FIG. 6f, respectively. The remaining mass of the scaffolds was determined by the gravimetric method and by measuring the overall area under the GPC curve; the curves of weight loss for both materials are similar in shape. Overall, compared to PLGAac, PLGAes degraded more slowly, but underwent chemical breakdown during degradation, showing immediate reduction in molecule weight and more rapid loss of nanofiber morphology.

Spatially-Controlled Release of Small Molecules from PCL-PLGAac and PCLPLGAes Scaffolds

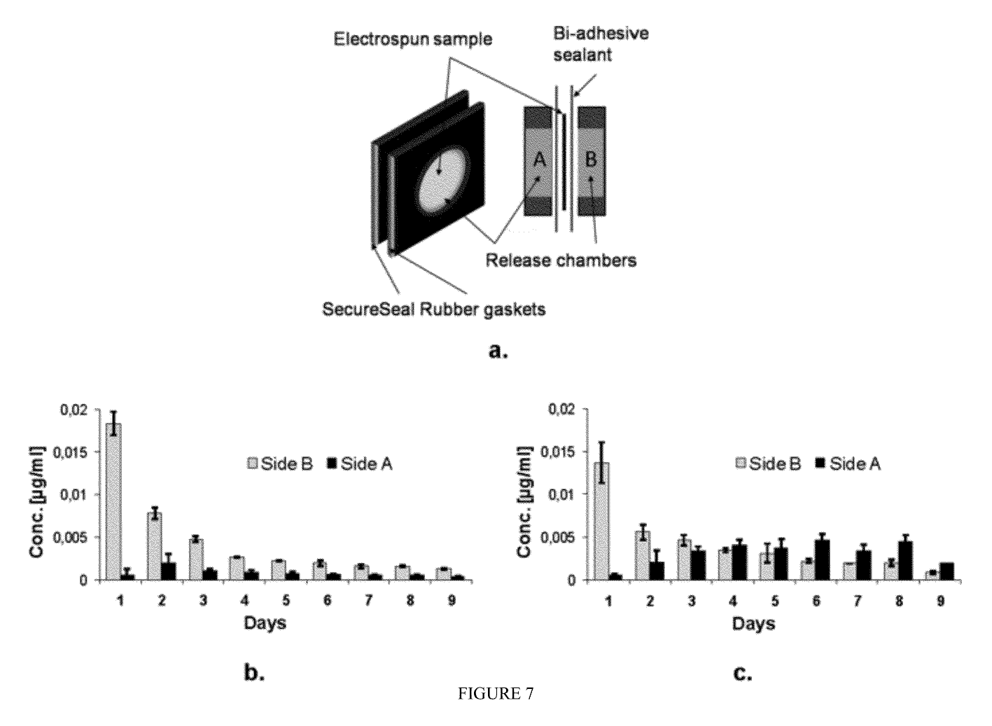

To assess the potential use of PCL-PLGAac and PCL-PLGAes materials for spatially controlled release of active molecules, release experiments were performed using Rh123. PCL and PLGA/Rh123 solutions were electrospun to prepare scaffolds following the pattern described in FIG. 1. Release studies were carried out in custom-made diffusion chambers illustrated in FIG. 7a. The two sealed reservoirs filled with phosphate buffer solution were placed on the opposite sides of the electrospun scaffolds. The PCL side of the scaffold was set to face the buffer reservoir chamber on Side “A”; the Rh123-impregnated PLGA side was placed in contact with the buffer reservoir on Side “B”. Daily measurement results of the Rh123 dye concentrations in both reservoirs were shown in FIG. 7. FIG. 7b demonstrated that the majority of the encapsulated fluorescent molecules were released from the PLGAac side. Thus, the electrospun PCL-PLGAac scaffolds demonstrated the capability of confining sustained molecule release to one side of the scaffolds. The separation efficiency of Rh123 between the two surfaces (S_{Rh123}_{_{—}}_{B-A}) of the PCL-PLGAac scaffolds was 82±5% during the 9-day release experiment. On the contrary, PCL-PLGAes samples displayed a mixed release behavior. FIG. 7c showed that more molecules were released from the PCL side, when compared to PCL-PLGAac. In addition, after day 3, the molecule concentration measured on the PCL side exceeded that on the PLGAes side. This resulted in an overall separation efficiency of around 50%; thus, no spatial separation of Rh123 between the two surfaces of the PCL-PLGAac scaffolds was found.

Based on the degradation and initial release experiment results, the PCL-PLGAac scaffold was used for the release of biomolecules.

Spatially-Controlled Protein Release from PCL-PLGAac Scaffolds

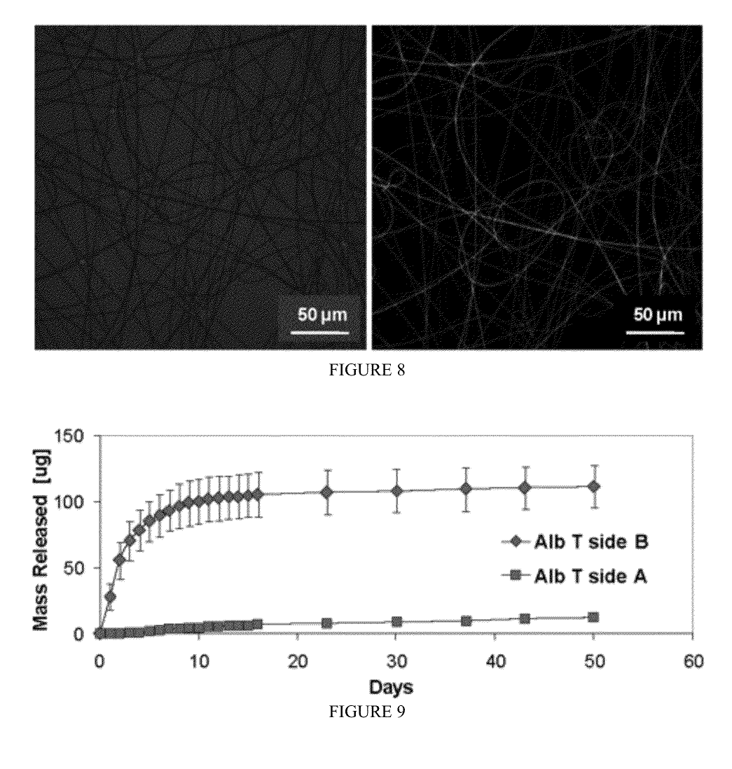

A variety of micropatterned composite scaffolds made up of PCL nanofibers and protein-impregnated PLGAac nanofibers were designed to demonstrate the versatile use of nanofiber micropattern for sustained release of one or two molecules with control in space and/or time scale. PLGAac nanofibers impregnated with fluorescently-labeled albumins were prepared by one-phase, solution electrospinning Albumin was dissolved directly in the polymeric solution prior to electrospinning and appeared to be homogeneously distributed in the PLGAac nanofibers (FIG. 8).

### Spatially-Controlled, Sustained Release of a Protein

FIG. 9 demonstrates the cumulative release of AlbT from the PCL-PLGAac electrospun scaffolds. The scaffolds were prepared according to the micropattern presented in FIG. 2. AlbT was impregnated in the PLGAac nanofibers. The scaffolds were characterized by a nanofiber gradient and thus AlbT gradient. Results showed that sustained release of AlbT was highly confined to the PLGAac side of the scaffolds (Side B), and the release of AlbT from the PCL surface (Side A) was limited. The separation efficiency for AlbT between side B and side A of PCL-PLGAac scaffolds was 90±4%, higher than the efficiency for smaller molecules (i.e. fluorophores). Results also showed that approximately 88% of the total amount of entrapped AlbT was released during the 50-day experiment. This estimation was made using an overall PLGAac content in the scaffold equal to 48%, as determined from DSC analysis of this specific AlbT-loaded PCL-PLGAac scaffold. Approximately 20% of the total loaded proteins were released in the first 24 hours, 80% were released in 9 days. After day 9, the low-slope release curve indicated a small amount of sustained release at a constant rate (about 1% per week).

### Sustained Releases of Two Proteins to One Side

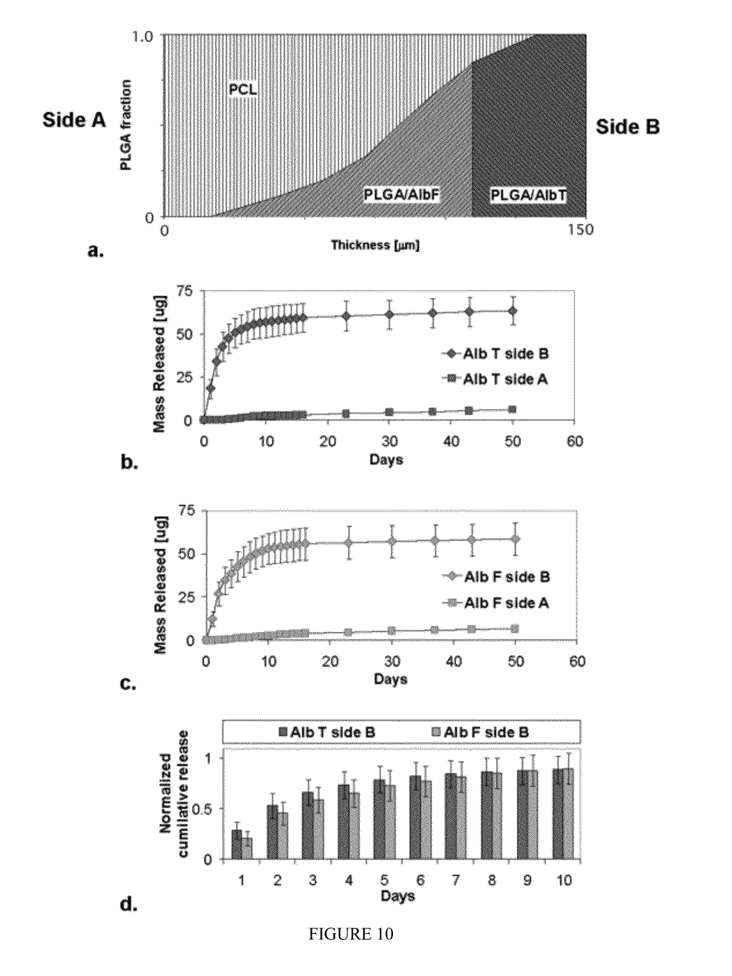

Using electrospun scaffolds with a compositional micropattern shown in FIG. 2d, the concurrent release of two proteins from the same side of the PCL-PLGAac composite scaffolds was demonstrated. As illustrated in FIG. 10a, PLGAac nanofibers in the inner part of the scaffolds were loaded with AlbF (green), while those in the outer region were loaded with AlbT (red). Results of cumulative release curves (FIG. 10b-c) showed that both AlbT and AlbF were preferentially released from one side of the scaffolds with high efficiency. The separation efficiencies of AlbT and AlbF were 91±4% and 87±5%, respectively. To compare the release kinetics of AlbT and AlbF during the first 10 days, the cumulative release on Side B was normalized with respect to the respective total amount of delivered proteins (FIG. 10d). The results suggested that AlbF initially was released at a slower rate, when compared to AlbT which was located closer to the surface.

### Sequential Releases of Two Molecules to One Side

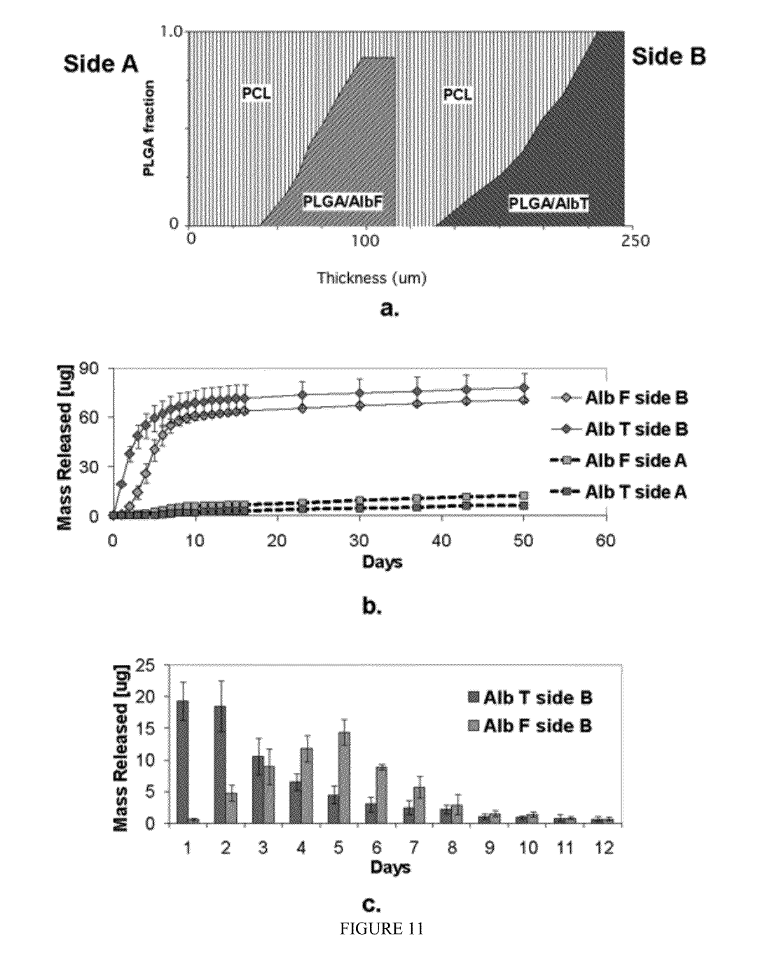

To achieve sequential releases of two molecules on the same side of the scaffold, the design strategy is based on the use of PCL, a hydrophobic, slowly degradable polymer, as a diffusion-barrier to segregate the molecules. Additionally, using PCL as the matrix to slow down the delivery, electrospun composite scaffolds with dual PLGA gradient patterns were designed for the sequential release of two proteins. The materials were prepared by continuous electrospinning in sequence of a 50 μm thick layer of PCL nanofibers, a mixed layer of PCL and AlbF-loaded PLGA nanofibers with varied PCL/PLGA ratios, a 25 μm-thick transitional layer of PCL nanofibers, and a mixed layer of PCL and AlbT-loaded PLGA nanofibers with varied PCL/PLGA ratios. FIG. 11a shows a schematic illustration of the pattern. The micro-pattern was characterized by an equal amount of AlbF-loaded PLGA and AlbT-loaded PLGA, and consequently, an equal amount of AlbF and AlbT. The overall composition was 55% (wt) of PCL, 22.5% (wt) of AlbF-loaded PLGA and 22.5% (wt) of AlbF-loaded PLGA.

FIG. 11b showed the cumulative release profiles of AlbF and AlbT released from both sides of the scaffold. It was found that both AlbF and AlbT were delivered preferentially to Side B. The separation efficiencies of AlbF and AlbT between side B and side A were 0.92±0.03 and 0.85±0.04, respectively. The scaffold was highly efficient in spatially confining the delivery of both AlbT and AlbF, though the spatial confinement was more efficient for AlbT than for Alb F. AlbF was released mainly on side B, although it was originally localized in a region close to the center of the scaffold. The small difference in the PCL content on the respective side of the PLGA/AlbF nanofiber regions determined the direction of AlbF protein delivery. This result further confirmed the important role of the PCL content in segregating proteins released from molecule-impregnated PLGA fibers to achieve spatially-controlled delivery. Daily net release of AlbF and AlbT to side B in the first 12 days were shown in c. The release kinetics of AlbF and AlbT are different from the study on dual protein release above. The net release of AlbT peaked on day 1 and day 2, while the net release of AlbF peaked on day 5.

### Sustained Releases of Two Molecules Respectively to the Opposite Sides

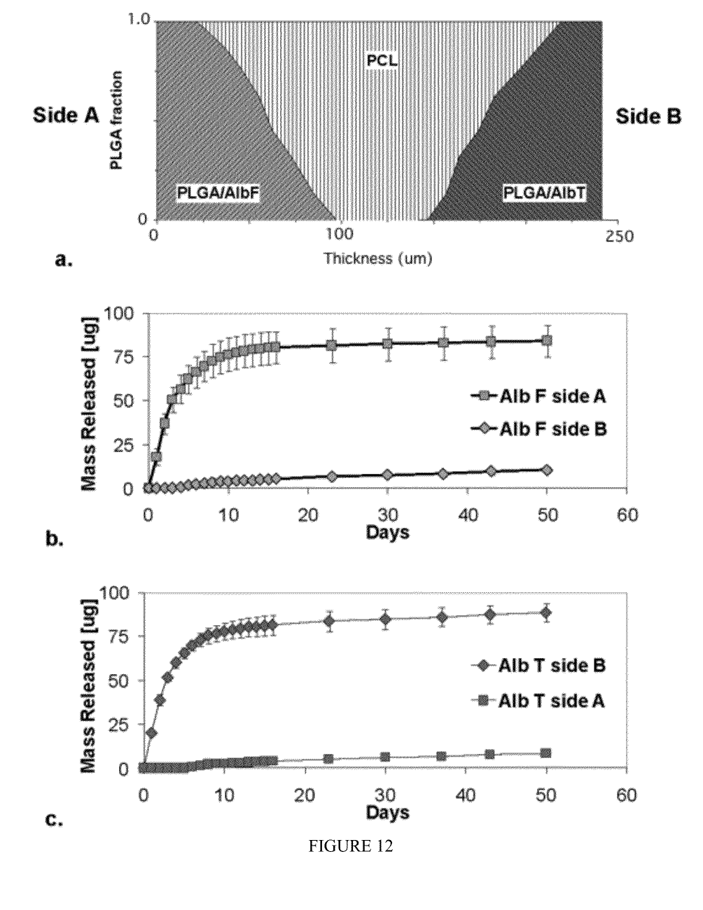

The micropattern of compositional gradient was further used as building-blocks of a more complex scaffold design for bi-directional release control purposes. As illustrated in FIG. 12a, the new pattern was generated by duplicating gradient patterns on both sides symmetric with respect to PCL. Herein, PLGAac was used to encapsulate either AlbF or AlbT. The designed pattern consisted of a 25-μm-thick layers of PLGAac electrospun nanofibers loaded with different fluorophore-tagged molecules on both surfaces, a 50 μm layer of PCL nanofibers in the core of the scaffold, and two PCL-PLGAac inter-layers characterized by a similar compositional gradient pattern. As a result, a precisely-controlled distribution of two biomolecules was generated across the scaffold thickness. Results from molecule release showed that AlbF and AlbT were selectively released from the opposite sides of the PLGAac-PCL-PLGAac composite scaffolds. The cumulative release curves for AlbF and AlbT are shown in FIG. 12b and FIG. 12c, respectively. AlbF was predominantly delivered to the reservoir on Side A, while AlbT was released to that on Side B. The separation efficiency of AlbF between Side A and Side B (S_{AlbF}_{_{—}}_{A-B}) was equal to 91±4%, while the separation efficiency of AlbT between Side B and Side A (S_{AlbT}_{_{—}}_{B-A}) was equal to 92±3%. Therefore, the two proteins were highly confined when delivered respectively to the opposite of the scaffold.

### Discussion

The present inventors have developed and characterized a platform of hierarchically-structured electrospun biomaterial scaffolds, which use the design of tunable nanofiber micropatterns to control spatial and temporal releases of one or multiple molecules. The molecule release was illustrated by the scaffolds with micropatterns characterized by complementary density gradients of distinct nanofibers. The scaffolds comprise PLGA fibers, an “active” molecule-releasing component that impregnates and releases model molecules, and PCL fibers, a “passive” component that sequesters molecule release while provides structural and mechanical integrity. Various compositional patterns were designed and imaged with confocal microscopy which demonstrated good correlations between designed and actual patterns. The different behaviors of PLGAac and PLGAes during degradation lead to their difference in controlled molecule release in our micropatterned scaffolds. Compared to PCL-PLGAes scaffolds, PLGAac nanofibers in the PCL-PLGAac scaffolds were more generally stable showing gradual and steady increase in the fiber diameter during hydrolytic degradation, and thus the separation efficiency, a measure of spatial confinement of molecule delivery, was much higher. Thus, spatially- and temporally-defined releases of proteins were both accomplished with PCL-PLGAac scaffolds. Using the graded composition and the interplay between PCL and PLGAac, it was shown that the hierarchically-structured composites were capable of sustaining selective release of small molecules or larger proteins on one side of the scaffold.

### Selection of PLGA Material for Controlled Release

PLGA is a biodegradable polymer that has been used in a wide range of medical implants, tissue engineering products and drug delivery devices, because of its slow degradation, good biocompatibility and mechanical property [31]. Both PLGAac and PLGAes are often used for preparing degradable scaffolds or controlled release materials, were largely different in physiochemical properties. But no previous studies have examined the influence of their physiochemical properties on their nanofiber morphology alterations during degradation and their capability of encapsulating and releasing molecules. It appears that the differences in the evolution of their nanofiber morphology could be attributed to the polymer state transition as determined from the DSC thermo-analysis. PLGAes nanofibers changed significantly since the beginning of degradation, lost fibrous features, and turned into a dense, continuous polymeric film in a week. Conversely, PLGAac nanofibers showed delayed morphological changes to a much later stage of degradation. Without being bound by any theory, it is believed that the rapid morphological evolution of PLGAes nanofibers is due to the fact that the onset point of the glass transition transformation for PLGAes (35.2° C.) was lower than the experimental/physiological temperature (37° C.). Although the glass transition temperature of PLGAes as measured from the inflection point of the DSC curve was 38.5° C., vast regions of PLGAes nanofibers undergo glass-rubber transitions at 37° C. Due to the phase transition, highly mobile polymer chains in the rubbery state allow polymer relaxation and water uptake. Acting as the plasticizer, the water molecules could contribute to further reduction of the glass transition temperature of PLGAes. PLGAes also underwent chemical breakdown during degradation, showing immediate reduction in molecule weight. Increased mobility of polymer chains caused nanofibers to start soldering together at their junctions and then coalesce to form a continuous film. In contrast, PLGAac whose glass transition onset point was at 37.8° C., resulted in more stable nanofiber morphology. The glass-rubber transition likely was deferred, preventing water uptake. The PLGAac fibrous structure thus retained for a longer time, showing open, interconnected pores during degradation. The microstructural characteristics resulted from degradation likely contributed to the different behaviours of PCL-PLGAes and PCL-PLGAac in controlling release of molecules impregnated in the PLGA fibers. The early formation of continuous, non-porous PLGAes layer might reduce the diffusion of biomolecules toward the PLGA surface, resulting in no confined release of Rh123 from PCL-PLGAes scaffolds after day 2, while PCL-PLGAac scaffolds maintained sustained, spatially-controlled molecule release over a 6-week long period. Without being bound by any theory, it is believed that molecule release from the nanofiber composite scaffold is largely determined by the degradation behaviors and morphological changes of PLGA fibers.

### Sustained, Spatially-Confined Molecule Release

Prolonged study on the release of albumins from PCL-PLGAac composite scaffolds demonstrated fast release in the first 9 days and steady but limited amount of sustained release over the rest of the experimental period up to 50 days. In particular, the release profile of albumin showed modest burst release (˜20% of total amount) on the first day. This burst release and subsequent fast release could be attributed to: (a) the nanofiber gradient pattern, since higher density of molecule-impregnated fibers were closer to the surface for earlier release, and (b) the molecule distribution in the nanofiber, since direct dissolution of molecules in the pre-spinning organic solution could result in a non-uniform distribution of molecules with a large portion segregated at the nanofiber surface [32]. Therefore, modifications of temporal release kinetics can be achieved in part by altering either the fiber pattern or the molecule encapsulation in the nanofiber. Encapsulation techniques other than one-phase blending can result in more steady release kinetics [33], [34]. Different from the molecule diffusion in the initial stage, the prolonged slow release in the later stage of the assay can be attributed to the polymer degradation behavior.

In addition to sustained release over the time, release studies with small molecules, one or multiple larger proteins all demonstrated high separation efficiency of molecule release on the side with higher molecule concentration or less PCL content. Diffusion to the other side was limited to around 10%. Therefore, the distribution of molecule-impregnated PLGAac nanofibers was effectively translated to sequestration of molecule release. The compositional gradient across the scaffold thickness confined molecule releases in the proximity of a surface of the scaffold, through the use of diffusion-limiting PCL material. Various release kinetics of combined biomolecules can be achieved by modulating the nanofiber micropattern and the composition profile including the thickness of the PCL layer, thickness and composition of transitional layers.

### Utility

A new generation of scaffolding materials for tissue engineering and regenerative medicine seeks to combine the material scaffolding function such as structural guidance and mechanical support with the regenerative function such as controlled molecule release to guide cell dynamics and organization [21]. Important to the success of in vivo and clinical studies is the potential of cells to grow healthy, functional tissues while avoid abnormal tissues or lesions, which underlines the important role of the accurate presentation modality of signaling molecules. In addition to the use of interpenetrating heterogeneous nanofiber network to meet diverse mechanical needs [14], the present invention shows that the use of heterogeneous nanofiber patterns in constructing scaffolding biomaterial can address various signaling needs for tissue formation. Because tissue regeneration is a highly regulated process involving differentiation and spatial organization of multiple cell types in 3D, sustained release of cell-specific molecules in a spatially-defined arrangement, as disclosed herein, can spatially guide cells and their commitment to regeneration. Uniform presentation of a regenerative molecule may not satisfy regeneration needs in many situations [29], because overexpression of a regenerative molecule or its presentation at a wrong place or a wrong time may lead to abnormal or diseased tissues. For example, PDGF is an important growth factor stimulating vascular SMC ingrowth and proliferation and thus is used for vascular tissue engineering, but it is well known as a major contributor to vascular intima dysfunction leading to intima hyperplasia and stenosis in vascular grafts. In some circumstances, biomaterials need to interface different tissues to stimulate simultaneous regeneration of these tissue functions, and thus different molecules might be required to release on opposite sides for the interface biomaterial implant [35]. The present invention discloses the potential of micropatterned nanofiber composite scaffolds using model fluorescent-labeled molecules or proteins, the material platform developed here can be applied to a wide range of applications, including therapeutic implants, tissue engineering, and drug delivery. In these applications, spatial confinement and temporal control of various molecules are important to the improvement of therapeutic efficacy and the reduction of biomolecule toxicity or side effects.

The gradient pattern shown in the material designs disclosed herein can be used as a building-block to design more complex molecule-fiber structures for controlling the release kinetics and direction of multiple biomolecules. To achieve more complex structures or designs, one can take advantage of the present inventors' programmable fabrication process which involves separate control over the fabrication of different nanofibers and their positions in 3D. Various release kinetics of combined biomolecules can be achieved by modulating the nanofiber micropattern and the composition profile including the thickness of the PCL layer, thickness and composition of transitional layer.

Regarding simultaneous regulation over release kinetics and direction of multiple molecules from regenerative scaffolds, the nanofiber-based materials disclosed herein can more readily program the desired molecule release kinetics for regeneration of mechanically strong tissues such as artery, when compared to previous studies that accomplish spatiotemporal releases for angiogenesis [36]. For instance, a regenerative artery substitute might benefit from the separate use of VEGF, PDGF and TGF-β with controlled release kinetics and direction. A scaffold releasing VEGF and PDGF respectively to the luminal and abluminal surfaces of an artery substitute might simultaneously promote the formation of an endothelial layer on the lumen while inducing migration of smooth muscle cells on the other side. Additionally, PDGF guide the migration of smooth muscle cells, while TGF-β is able to accelerate their proliferation, and thus a sequential release of PDGF and TGF-β with proper timing may promote regeneration of arterial media. Therefore, a scaffold with respective controls over these three molecules through the use of the gradient design as scaffold building blocks can significantly promote the process of arterial regeneration. Moreover, the optimal release kinetics for tissue regeneration in vitro or in vivo is largely unexplored and far from being well known. Compositions and methods for producing the same disclosed herein can also be used to identify an optimal condition from a large variety of release patterns and to study the underlying molecule mechanism.

Use of more environment sensitive molecules, such as growth factors whose bioactivity is an important issue in the controlled release, can also be used in various applications. Electrospinning of molecule-impregnated nanofibers has previously been used to release bioactive growth factors. Also, other molecule encapsulation methods have been developed for electrospun materials to retain protein bioactivity [32], which might be used in conjunction with the techniques disclosed herein.

### CONCLUSION

Disclosed herein are micropatterned nanofiber composites made from biodegradable PCL and PLGA. Compositions of the invention are useful in sustained, spatiotemporally-controlled release of one or multiple bioactive molecules, e.g., proteins or small molecules. The hierarchically-structured composite scaffolds can be used to define 3D dynamic microenvironments and can be used in a variety of applications including, but not limited to, tissue engineering and therapeutic device.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references disclosed herein are incorporated by reference in their entirety.

### REFERENCES

1. Whatley B R, Kuo J, Shuai C, Damon B J, Wen X. Fabrication of a biomimetic elastic intervertebral disk scaffold using additive manufacturing. Biofabrication. 2011; 3(1):015004.
2. Baker B M, Mauck R L. The effect of nanofiber alignment on the maturation of engineered meniscus constructs. Biomaterials. 2007; 28(11):1967-77.
3. Nerurkar N L, Baker B M, Sen S, Wible E E, Elliott D M, Mauck R L. Nanofibrous biologic laminates replicate the form and function of the annulus fibrosus. Nature materials. 2009; 8(12):986-92.
4. Moffat K L, Wang I-NE, Rodeo S A, Lu H H. Orthopedic interface tissue engineering for the biological fixation of soft tissue grafts. Clinics in sports medicine. 2009; 28(1):157-76.
5. Li W-J, Jiang Y J, Tuan R S. Cell-nanofiber-based cartilage tissue engineering using improved cell seeding, growth factor, and bioreactor technologies. Tissue engineering. Part A. 2008; 14(5):639-48.
6. Drilling S, Gaumer J, Lannutti J. Fabrication of burst pressure competent vascular grafts via electrospinning: effects of microstructure. Journal of biomedical materials research. Part A. 2009; 88(4):923-34.
7. Yang X, Shah J D, Wang H. Nanofiber enabled layer-by-layer approach toward three-dimensional tissue formation. Tissue engineering. Part A. 2009; 15(4):945-56.
8. Griffin J, Delgado-Rivera R, Meiners S, Uhrich K E. Salicylic acid-derived poly(anhydride-ester) electrospun fibers designed for regenerating the peripheral nervous system. Journal of biomedical materials research. Part A. 2011; 97(3):230-42.
9. Slater S C, Beachley V, Hayes T, Zhang D, Welsh G I, Saleem M A, et al. An in vitro model of the glomerular capillary wall using electrospun collagen nanofibres in a bioartificial composite basement membrane. PloS one. 2011; 6(6):e20802.
10. Karageorgiou V, Tomkins M, Fajardo R, Meinel L, Snyder B, Wade K, et al. Porous silk fibroin 3-D scaffolds for delivery of bone morphogenetic protein-2 in vitro and in vivo. Journal of biomedical materials research. Part A. 2006; 78(2):324-34.
11. Mieszawska A J, Kaplan D L. Smart biomaterials—regulating cell behavior through signaling molecules. BMC biology. 2010; 8:59.
12. Zhang X, Reagan M R, Kaplan D L. Electrospun silk biomaterial scaffolds for regenerative medicine. Advanced drug delivery reviews. 2009; 61(12):988-1006.
13. Chew S Y, Wen J, Yim E K F, Leong K W. Sustained release of proteins from electrospun biodegradable fibers. Biomacromolecules. 2005; 6(4):2017-24.
14. Bonani W, Maniglio D, Motta A, Tan W, Migliaresi C. Biohybrid nanofiber constructs with anisotropic biomechanical properties. Journal of Biomedical Materials Research Part B-Applied Biomaterials. 2011; 96B(2):276-86.
15. Ionescu L C, Lee G C, Sennett B J, Burdick J A, Mauck R L. An anisotropic nanofiber/microsphere composite with controlled release of biomolecules for fibrous tissue engineering. Biomaterials. 2010; 31(14):4113-20.
16. Wei G, Jin Q, Giannobile W V, Ma P X. Nano-fibrous scaffold for controlled delivery of recombinant human PDGF-BB. Journal of Controlled Release. 2006; 112(1):103-10.
17. Hile D D, Pishko M V. Solvent-free protein encapsulation within biodegradable polymer foams. Drug delivery. 2004; 11(5):287-93.
18. Guan J, Stankus J J, Wagner W R. Biodegradable elastomeric scaffolds with basic fibroblast growth factor release. Journal of Controlled Release. 2007; 120(1-2):70-8.
19. Nie T, Baldwin A, Yamaguchi N, Kiick K L. Production of heparin-functionalized hydrogels for the development of responsive and controlled growth factor delivery systems. Journal of Controlled Release. 2007; 122(3):287-96.
20. Sokolsky-Papkov M, Agashi K, Olaye A, Shakesheff K, Domb A J. Polymer carriers for drug delivery in tissue engineering. Advanced drug delivery reviews. 2007; 59(4-5):187-206.
21. Biondi M, Ungaro F, Quaglia F, Netti P A. Controlled drug delivery in tissue engineering. Advanced Drug Delivery Reviews. 2008; 60(2):229-42.
22. Discher D E, Mooney D J, Zandstra P W. Growth Factors, Matrices, and Forces Combine and Control Stem Cells. Science. 2009; 324(5935):1673-7.
23. Lee K, Silva E A, Mooney D J. Growth factor delivery-based tissue engineering: general approaches and a review of recent developments. Journal of the Royal Society, Interface. 2011; 8(55):153-70.
24. Salvay D M, Shea L D. Inductive tissue engineering with protein and DNAreleasing scaffolds. Molecular bioSystems. 2006; 2(1):36-48. 25. Chen F-M, Zhang M, Wu Z-F. Toward delivery of multiple growth factors in tissue engineering. Biomaterials. 2010; 31(24):6279-308.
26. Sy J C, Davis M E. Delivering regenerative cues to the heart: cardiac drug delivery by microspheres and peptide nanofibers. Journal of cardiovascular translational research. 2010; 3(5):461-8.
27. Gandhi M, Srikar R, Yarin A L, Megaridis C M, Gemeinhart R A. Mechanistic examination of protein release from polymer nanofibers. Molecular pharmaceutics. 2009; 6(2):641-7.
28. Ainslie K M, Lowe R D, Beaudette T T, Petty L, Bachelder E M, Desai T A. Microfabricated devices for enhanced bioadhesive drug delivery: attachment to and small-molecule release through a cell monolayer under flow. Small (Weinheim an der Bergstrasse, Germany). 2009; 5(24):2857-63.
29. Silva E A, Mooney D J. Effects of VEGF temporal and spatial presentation on angiogenesis. Biomaterials. 2010; 31(6):1235-41.
30. Martin T A, Herman C T, Limpoco F T, Michael M C, Potts G K, Bailey R C. Quantitative photochemical immobilization of biomolecules on planar and corrugated substrates: a versatile strategy for creating functional biointerfaces. ACS applied materials & interfaces. 2011; 3(9):3762-71.
31. Puppi D, Piras A M, Detta N, Dinucci D, Chiellini F. Poly(lactic-co-glycolic acid) electrospun fibrous meshes for the controlled release of retinoic acid. Acta biomaterialia. 2010; 6(4):1258-68.
32. Zhang Y Z, Wang X, Feng Y, Li J, Lim C T, Ramakrishna S. Coaxial electrospinning of (fluorescein isothiocyanate-conjugated bovine serum albumin)-encapsulated poly(epsilon-caprolactone) nanofibers for sustained release. Biomacromolecules. 2006; 7(4):1049-57.
33. Huang Z M, He C L, Yang A Z, Zhang Y Z, Hang X J, Yin J L, et al. Encapsulating drugs in biodegradable ultrafine fibers through co-axial electrospinning Journal of Biomedical Materials Research Part A. 2006; 77A(1):169-79.
34. Xu X, Yang L, Xu X, Wang X, Chen X, Liang Q, et al. Ultrafine medicated fibers electrospun from W/O emulsions. Journal of controlled release: official journal of the Controlled Release Society. 2005; 108(1):33-42.
35. Phillips J E, Burns K L, Le Doux J M, Guldberg R E, García A J. Engineering graded tissue interfaces. Proceedings of the National Academy of Sciences of the United States of America. 2008; 105(34):12170-5.
36. Jain R K. Molecular regulation of vessel maturation. Nature Medicine. 2003; 9(6):685-93.

What is claimed is:

1. A biocompatible composition comprising:
a first surface;
a second surface; and
at least one polymeric mixture layer comprising a plurality of biodegradable polymers, wherein said polymeric mixture layer comprises a first biodegradable polymer and a second biodegradable polymer, and wherein the relative amount of said first biodegradable polymer or said second biodegradable polymer increases within said polymeric mixture layer in the direction from said first surface to said second surface, and the relative amount of said other biodegradable polymer decreases in said polymeric mixture layer in the direction from said first surface to said second surface, and wherein the rate of degradation of said first biodegradable polymer is different from the rate of degradation of said second biodegradable polymer.

2. The biocompatible composition of claim 1 further comprising a plurality of said polymeric mixture layers.

3. The biocompatible composition of claim 2 further comprising a barrier polymeric layer between said polymeric mixture layers.

4. The biocompatible composition of claim 3, wherein said barrier polymeric layer comprises said first biodegradable polymer or said second biodegradable polymer.

5. The biocompatible composition of claim 1, wherein said first biodegradable polymer in said polymeric mixture layer comprises a first bioactive molecule.

6. The biocompatible composition of claim 5, wherein said first bioactive molecule is adsorbed in, attached to, or encapsulated within said first biodegradable polymer, or a combination thereof.

7. The biocompatible composition of claim 6, said first biodegradable polymer comprises covalently bonded bioactive molecule, non-covalently bonded bioactive molecule, or a combination thereof.

8. The biocompatible composition of claim 5, wherein said first bioactive molecule comprises a protein, a drug, an oligonucleotide, a chemical signaling agent, or a mixture thereof.

9. The biocompatible composition of claim 5, wherein the relative amount of said first biodegradable polymer decreases within said polymeric mixture layer in the direction from said first surface to said second surface.

10. The biocompatible composition of claim 9, wherein the amount of said first bioactive molecule released towards said first surface during biodegradation is higher than the amount of said first bioactive molecule released towards said second surface.

11. The biocompatible composition of claim 10, wherein the ratio of the amount said first bioactive molecule released towards said first surface compared to the amount of said first bioactive molecule released towards said second surface during biodegradation is at least 2:1.

12. The biocompatible composition of claim 1, wherein said polymeric mixture layer comprises a nanofiber mixture of said first and second biodegradable polymers.

13. The biocompatible composition of claim 1, wherein said biocompatible composition is porous.

14. A method for controlling the relative direction of and/or the relative rate of bioactive material released in a biodegradable polymer, said method comprising:
providing the bioactive material within a biocompatible polymer comprising:
a first surface;
a second surface; and
at least one polymeric mixture layer comprising a plurality of biodegradable polymers,
wherein the polymeric mixture layer comprises
a first biodegradable polymer that comprises the bioactive material; and
a second biodegradable polymer,
and wherein the relative amount of said first biodegradable polymer or said second biodegradable polymer increases within said polymeric mixture layer in the direction from said first surface to said second surface, and the relative amount of said other biodegradable polymer decreases in said polymeric mixture layer in the direction from said first surface to said second surface, and wherein the rate of degradation of said first biodegradable polymer is different from the rate of degradation of said second biodegradable polymer.
